(19) United States
(12) Patent Application Publication
(10) Pub. No.: US 2005/0222613 A1
(43) Pub. Date: Oct. 6, 2005

(54) Wound closure device and method for vitrectomy

(76) Inventor: Edwin Ryan, St. Paul, MN (US)

Correspondence Address: Schwegman, Lundberg, Woessner & Kluth, P.A., P.O. Box 2938, Minneapolis, MN, 55402, US

(21) Appl. No.: 11/052,452

(22) Filed: Feb. 7, 2005

Related U.S. Application Data
(60) Provisional application No. 60/542,212, filed on Feb. 5, 2004.

(51) Int. Cl. A61B 17/10; A61D 1/00

(52) U.S. Cl. 606/213

(57) ABSTRACT

A method and device for wound closure is provided. One advantage of wound closure devices and methods shown includes a simple and inexpensive alternative to suturing incisions in eyes to prevent leakage. Another advantage includes a plug formed from a biocompatible material that degrades over time to allow healing of the incision.

### RELATED APPLICATIONS

This application claims the benefit of U.S. Provisional Application Ser. No. 60/542,212, filed Feb. 5, 2004, which provisional application is incorporated herein by references.

### TECHNICAL FIELD

This invention relates to wound closure devices and methods to facilitate healing after an incision. Specifically, this invention relates to wound closure devices and methods for use in conjunction with vitreous surgery.

### BACKGROUND

Vitreous surgery was first performed in 1971, and involves the removal of the vitreous gel from the posterior aspect of the eye for treatment of a variety of disease states, including vitreous hemorrhage, macular disorders, retinal detachment, and many others. One common procedure involves the use of 3 incisions peripheral to the cornea to access the vitreous cavity. One port is used for infusion, one for illumination, and the third for suction/cutting instruments, as well as picks, scissors, forceps and others.

During some vitreous surgical procedures the small incision, or wound, leaks after the surgical instrument is removed from the incision. One common procedure to reduce or stop leakage is to suture the incision. Suturing in such circumstances can be difficult and tedious for the surgeon. As with any difficult procedure there is some degree of risk to the patient as well.

What is needed is a device an method to reduce leakage through incisions following vitreous surgery that is easier, and less expensive. What is also needed is a device an method to reduce risk to the patient.

### BRIEF DESCRIPTION OF THE DRAWINGS

FIG. 1 shows a wound closure device according to an embodiment of the invention.

FIG. 2 shows another wound closure device according to an embodiment of the invention.

FIG. 3A shows a portion of a procedure according to an embodiment of the invention.

FIG. 3B shows another portion of a procedure according to an embodiment of the invention.

FIG. 4 shows a flow chart of one method according to an embodiment of the invention.

### DETAILED DESCRIPTION

In the following detailed description, reference is made to the accompanying drawings which form a part hereof, and in which is shown, by way of illustration, specific embodiments in which the invention may be practiced. In the drawings, like numerals describe substantially similar components throughout the several views. These embodiments are described in sufficient detail to enable those skilled in the art to practice the invention. Other embodiments may be utilized and structural, mechanical, logical changes, etc. may be made without departing from the scope of the present invention.

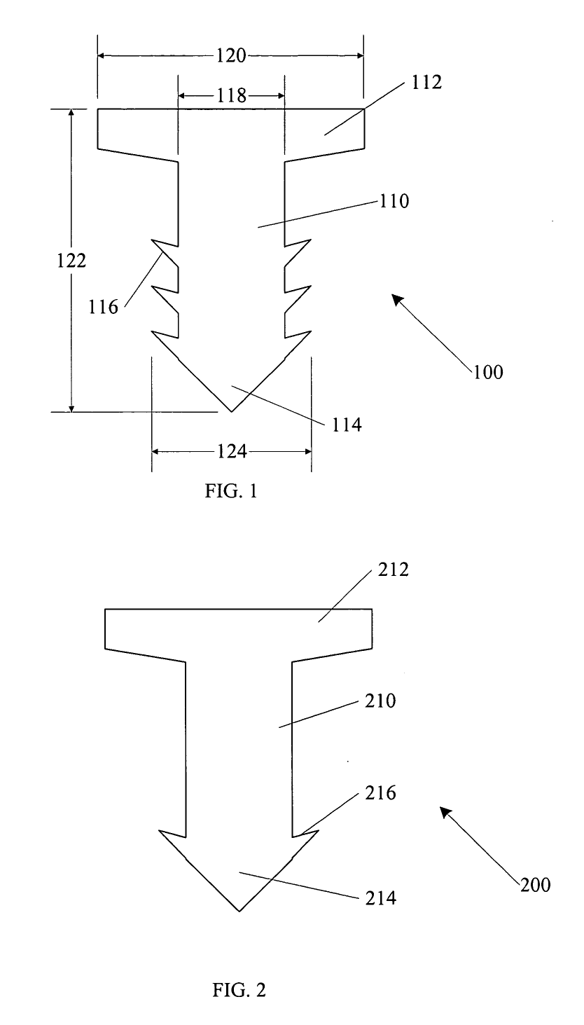

FIG. 1 shows a plug 100 according to an embodiment of the invention. A body portion 110 is shown with a cover portion 112 and a tapered end portion 114. In the embodiment shown in FIG. 1, a number of sets of barbs 116 are included along a longitudinal portion of the body 110. Although a barb 116 is shown, other holding portions such as an enlarged region are also within the scope of the invention. In one embodiment the plug is dimensioned to close a 25 gauge incision as will be described in more detail below. In one embodiment the plug is dimensioned to close a 23 gauge incision. Although 25 and 23 gauge incisions are mentioned specifically, the invention is not so limited. One of ordinary skill in the art, having the benefit of the present disclosure will recognize that embodiments of the present invention are possible for a number of incision dimensions.

In one embodiment, a body portion diameter 118 includes 0.3 mm. In one embodiment, a barb diameter 124 includes 0.5 mm. In one embodiment, a cover portion diameter 120 include 0.8 mm. In one embodiment a plug length 122 includes 1.0 mm. As stated above, although specific dimensions are listed, other dimensions are possible for various incision geometries. Although circular portions are discussed with various diameters, other geometries such as hexagonal cross sections, oval cross sections, etc. adapted to reduce leakage in an incision are within the scope of the invention.

FIG. 2 shows a plug 200 according to an embodiment of the invention. A body portion 210 is shown with a cover portion 212 and a tapered end portion 214. At least one holding portion 216 such as a barb is included adjacent to the tapered end portion 214. In one embodiment, the plug 200 includes dimensions similar to the plug 100 shown in FIG. 1. As discussed above, other dimensions and geometries are also within the scope of the invention.

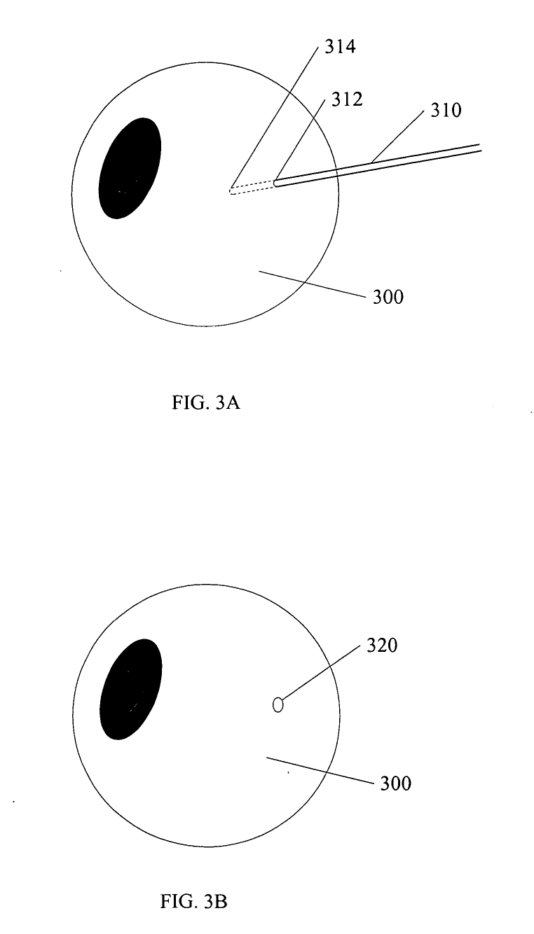

FIG. 3A shows a portion of a procedure utilizing wound closure devices as described above. An eye 300 is shown. During a procedure such as vitreous surgery, an instrument 310 is inserted into the eye through an incision 312 to reach a location 314 internal to the eye. Examples of instruments 310 include, but are not limited to fiber optic probes, laser guides, suction/cutting tools, forceps, scissors, etc.

FIG. 3B shows the eye 300 as shown in FIG. 3A after removal of the instrument 310. In one method, a plug 320 as described in embodiments above is inserted into the incision. The body portion of the plug fills the incision and reduces or prevents leakage from the incision. In one embodiment, a cover portion as described above is held against the surface of the eye 300, while a holding portion as described above retains the plug in the incision. In one embodiment, similar to FIG. 2, a holding portion 216 engages a bottom edge of the incision. In one embodiment, similar to FIG. 1, a plurality of holding portions 216 such as barbs engage either a bottom edge of the incision, or an inside portion of the incision. In one embodiment a holding portion dimension 124 as described above is dimensioned slightly larger than an incision dimension to provide the holding function as described above after resilient deformation of holding portions. For example, a 0.5 mm holding portion can be used with a 25 gauge incision (0.49 mm)

In one embodiment the plug is fabricated from a polyglycolic acid polymer. One example of a polyglycolic acid polymer include Vicryl™. In one embodiment, the plug is fabricated from collagen. Other materials are also within the scope of the invention. In one embodiment, the entire plug is integrally formed from a single material although the invention is not so limited. Desirable material/structural properties of the plug include sufficient stiffness to insert the plug into the incision, yet have the plug remain held within the incision during healing. Another desirable property of the plug includes a biocompatible plug that is non-toxic, does not cause inflammation, etc. Another desirable property of the plug includes a biodegradable material or similar property that allows the plug to dissolve, soften, or otherwise degrade over time. A desirable time frame for such degradation would be a few days, allowing the incision to heal, while still reducing or preventing leakage.

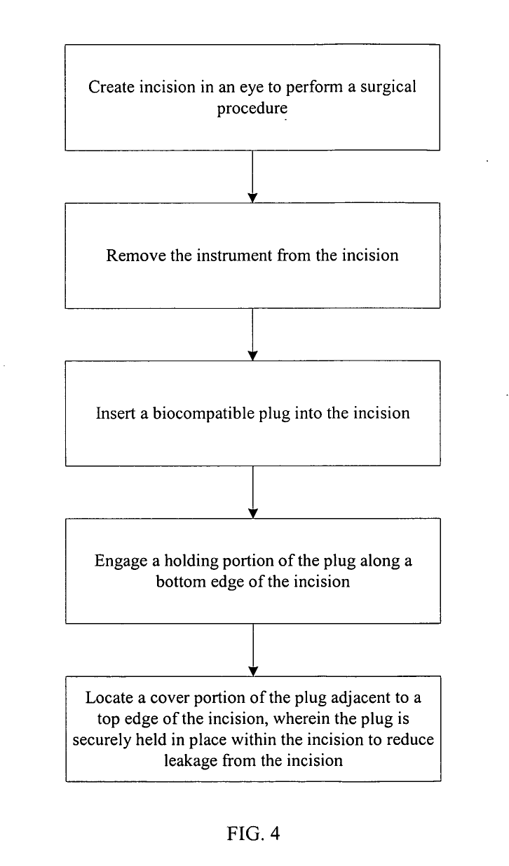

FIG. 4 shows one embodiment of an eye surgery method according to an embodiment of the invention. As shown, an incision is created in a patient's eye using an instrument during a surgical procedure. After the procedure is complete, the instrument is removed from the eye, leaving the incision. A biocompatible plug according to embodiments of the invention described in the present disclosure is inserted into the incision. One or more holding portions are engaged on or within the incision to hold the plug in place. A cover portion is further located adjacent to the incision on the surface of the eye in one embodiment to help reduce leakage, and to hold the plug in place.

### CONCLUSION

Using embodiments described above, a number of advantages are realized. One advantage of wound closure devices and methods described above includes a simple and inexpensive alternative to suturing incisions in eyes to prevent leakage. Another advantage includes a biocompatible material that degrades over time to allow healing of the incision.

Although selected advantages are detailed above, the list is not intended to be exhaustive. Although specific embodiments have been illustrated and described herein, it will be appreciated by those of ordinary skill in the art that any arrangement which is calculated to achieve the same purpose may be substituted for the specific embodiment shown. This application is intended to cover any adaptations or variations of the present invention. It is to be understood that the above description is intended to be illustrative, and not restrictive. Combinations of the above embodiments, and other embodiments will be apparent to those of skill in the art upon reviewing the above description. The scope of the invention includes any other applications in which the above structures and fabrication methods are used. The scope of the invention should be determined with reference to the appended claims, along with the full scope of equivalents to which such claims are entitled.

1. A method of closing an incision in an eye, comprising:
inserting a biocompatible plug into an incision in the eye;
engaging a holding portion of the plug with the incision; and
locating a cover portion of the plug adjacent to a top edge of the incision, wherein the plug is securely held in place within the incision to reduce leakage from the incision.

2. The method of claim 1, wherein inserting the biocompatible plug includes inserting a plug adapted for a 25 gauge instrument incision.

3. The method of claim 1, wherein inserting the biocompatible plug includes inserting a plug adapted for a 23 gauge instrument incision.

4. The method of claim 1, wherein engaging the holding portion of the plug includes engaging a deformable barbed fastener portion.

5. The method of claim 1, wherein inserting the biocompatible plug includes inserting a biodegradable plug.

6. A wound closure device, comprising:
a body portion dimensioned to substantially fill an incision in an eye;
at least one deformable barb to hold the wound closure device within the incision;
a cover portion to engage an outer portion of the eye around the incision;
wherein the wound closure device is formed from a material that is compatible with eye tissue, and degrades over a time period when the incision heals.

7. The wound closure device of claim 6, wherein at least one deformable barb includes a series of barbs along a longitudinal axis of the body portion.

8. The wound closure device of claim 6, wherein the body portion is dimensioned to substantially fill a 25 gauge incision in an eye.

9. The wound closure device of claim 6, wherein the body portion is demensioned to substantially fill a 23 gauge incision in an eye.

10. The wound closure device of claim 6, wherein the material of the wound closure device includes collagen.

11. The wound closure device of claim 6, wherein the material of the wound closure device includes a polyglycolic acid polymer.
